(19) United States
(12) Patent Application Publication
(10) Pub. No.: US 2008/0050725 A1
(43) Pub. Date: Feb. 28, 2008

(54) Methods of detecting DNA N-glycosylases, methods of determining N-glycosylase activity, and N-glycosylase assay kits

(75) Inventor: William K. Keener, Falling Waters, WV (US)

Correspondence Address: BATTELLE ENERGY ALLIANCE, LLC, P.O. BOX 1625, IDAHO FALLS, ID, 83415-3899, US

(73) Assignee: Battelle Energy Alliance, LLC

(21) Appl. No.: 11/511,627

(22) Filed: Aug. 28, 2006

(51) Int. Cl. C12Q 1/68

(52) U.S. Cl. 435/6

(57) ABSTRACT

The invention includes methods of detecting glycosylases. A test sample is mixed with substrate polynucleotide. A primer and a polymerase are added. An endonuclease is provided and a probe oligonucleotide sequence labeled with first and second labels is utilized for detection. The invention includes N-glycosylase assay methods. A test sample is mixed with substrate polynucleotide and formation of an abasic site is detected by forming a product that is complementary to a portion of the substrate sequence ending at the abasic site. The product is dissociated and is extended utilizing a polymerase. A probe is hybridized to the product and is cleaved. The invention includes synthetic substrates, transcription primers and probe molecules. The invention also includes an N-glycosylase detection kit including a substrate polynucleotide, an endonuclease and a dual-labeled probe having a fluorescent label and a quencher moiety.

### GOVERNMENT RIGHTS

The United States Government has certain rights in this invention pursuant to Contract No. DE-AC07-05ID14517 between the United States Department of Energy and Battelle Energy Alliance, LLC.

### TECHNICAL FIELD

The invention pertains to methods of detecting a DNA N-glycosylase and N-glycosylase assay methods. The invention additionally pertains to oligonucleotide probes, synthetic polynucleotides, compositions of matter containing oligonucleotides, and glycosylase detection kits.

### BACKGROUND OF THE INVENTION

Glycosylases are enzymes that catalyze hydrolysis of N-glycosylic bonds between a base and a sugar moiety of a nucleic acid resulting in an abasic site. N-glycosylase activity occurs on DNA substrates, whereas N-glycosidase activity occurs on RNA substrates, although a given enzyme may act on both DNA and RNA substrates. N-glycosylases having specificity can specifically depurinate or depyrimidate nucleic acid and in particular instances can have a particular recognition site within a nucleic acid sequence. Glycosylase activity can result in an abasic site at one or more location within a polynucleotide sequence resulting in an aldehyde group on the sugar residue and leaving an intact phosphodiester backbone.

The biological function of many DNA N-glycosylases is to remove bases which are improperly incorporated or damaged. Production of an abasic site in a template DNA can inhibit polymerase activity at the abasic site causing the polymerase to pause during DNA synthesis. An exemplary DNA glycosylase is uracil-DNA glycosylase which removes uracil from DNA.

In contrast to the repair function of DNA glycosylases, adenine-specific RNA N-glycosidases function to cause damage. Ribosome inactivating N-glycosidases typically remove an adenine residue from, or depurinate, ribosomal RNA. Exemplary N-glycosidases including ricin, saporin and gelonin have the ability to inactivate ribosomes by depurination of ribosomal RNA. Each of these enzymes is also able to remove adenine from DNA molecules (DNA N-glycosylase activity).

Due to their ability to remove purine bases from ribosomal RNA to inhibit or block protein synthesis, RNA N-glycosidases are potential bioterrorist agents. N-glycosidases such as ricin and abrin are bio-threats. Interestingly, known toxins such as gelonin, saporin, and ricin A chain (the enzyme portion of ricin) can be utilized for treatment or therapeutic purposes. Strategies have been developed where such “toxins” are coupled to large molecules that bind diseased cells to specifically deliver and target the toxin to such cells.

In both therapeutic situations and in detection of potential bioterrorist agents, it is important to have sensitive assays for N-glycosylase/glycosidase activity. However, conventional assay and detection methodology in this area are typically complex, often requiring large, specialized and/or highly sensitive equipment. The time and equipment involved in performing such conventional detection/activity determination render it difficult or impossible to perform such assays remotely or in the field. It is desirable to develop alternative N-glycosylase assay and detection methods.

### SUMMARY OF THE INVENTION

In one aspect the invention encompasses a method of detecting a glycosylase. A sample to be tested for the presence of a glycosylase is provided and is mixed with a substrate polynucleotide to form an initial mixture. An oligonucleotide primer and a polymerase are added to the initial mixture to form an assay mixture. An endonuclease is provided into the assay mixture. The assay mixture is contacted with a probe oligonucleotide sequence labeled with a first and second label.

In one aspect the invention encompasses an N-glycosylase assay method. A sample to be tested for N-glycosylase activity is mixed with substrate polynucleotide molecules. The presence of abasic sites produced on the polynucleotide molecule is detected by forming an oligonucleotide product that is complementary to a portion of the substrate polynucleotide sequence ending at the abasic site. The product is dissociated from the substrate polynucleotide and is extended utilizing a polymerase. A probe is hybridized to a portion of the oligonucleotide product and the probe is cleaved.

In one aspect the invention encompasses synthetic polynucleotide substrates, transcription primers and probe molecules.

In one aspect the invention encompasses an N-glycosylase detection kit. The kit includes a substrate polynucleotide having an N-glycosylase target sequence, an endonuclease and a probe having a fluorescent label at a first end of an oligonucleotide and a quencher moiety and the second end of the oligonucleotide.

### BRIEF DESCRIPTION OF THE DRAWINGS

Preferred embodiments of the invention are described below with reference to the following accompanying drawings.

FIG. 1 illustrates an exemplary synthetic substrate and primer in accordance with a first aspect of the invention.

FIG. 2 is a flow-chart diagram showing general methodology in accordance with one aspect of the invention.

FIG. 3 illustrates an initial phase of methodology in accordance with the invention performed in the absence (Panel A) and presence (Panel B) of an N-glycosylase.

FIG. 4, Panels A and B illustrate reaction events at a stage subsequent to that shown in FIGS. 3, Panels A and B respectively.

FIG. 5, Panels A and B illustrate reaction events subsequent to that shown in FIGS. 4 Panels A and B respectively.

FIG. 6, Panels A and B show reaction subsequent to that depicted in FIGS. 5, Panels A and B respectively.

FIG. 7, Panels A and B illustrate reaction events at a stage subsequent to that depicted in FIG. 6, Panels A and B, respectively.

FIG. 8, Panels A and B illustrate reaction events at a stage subsequent to that depicted in FIGS. 7, Panels A and B respectively.

FIG. 9 shows the results of fluorescent analysis of control endonuclease reaction study samples (nuclease free). Sample A is a control reaction performed in an absence of substrate oligonucleotide. Sample B is a control reaction performed utilizing an oligonucleotide substrate having a sequence as set forth in SEQ ID NO.:4. Sample C is a control endonuclease reaction performed utilizing a synthetic polynucleotide having a synthetic abasic site and the sequence set forth in SEQ ID NO.:10. Sample D shows the fluorescence of a control endonuclease reaction where the substrate has been substituted with a simulating product oligonucleotide (mimic) having the sequence set forth in SEQ ID NO.: 11. Sample E is a control sample performed utilizing a synthetic nicked template molecule having the sequence set forth in SEQ ID NO.: 16

FIG. 10 demonstrates the sensitivity of assays performed in accordance with a first aspect of the invention. The observable fluorescence represents varying percentage of conversion as determined utilizing 2.5 pmol total oligonucleotide made up of varying ratios of substrate oligonucleotide and synthetic product oligonucleotide with artificial abasic site (SEQ ID NO.:10).

FIG. 11 shows sub-picogram detection of uracil N-glycosylase. Sample A contains 34 pg uracil N-glycosylase (UNG), sample B contains 3.4 pg UNG, sample C contains 0.0 pg UNG, sample D contains 340 fg UNG, sample E contains 34 fg UNG, sample F contains 0.0 fg UNG, and sample G contains a 10% conversion equivalent control. The ‘*’ symbol denotes the detection limit.

FIG. 12 further illustrates sub-picogram detection of uracil N-glycosylase utilizing extended reaction times and additional thermocycles. Sample A contains 17 pg uracil N-glycosylase (UNG), sample B contains 1.7 pg UNG, sample C contains 0.0 pg UNG, sample D contains 170 fg UNG, sample E contains 17 fg UNG, sample F contains 0.0 fg UNG and sample G contains a 10% conversion equivalent reference sample. The ‘*’ symbol denotes the detection limit.

FIG. 13 shows the specificity of uracil N-glycosylase reactions for uracil-containing oligonucleotide substrates. Samples A-D were performed utilizing 2.5 pmol UNG oligonucleotide substrate(SEQ ID NO.:3) with samples A and B being performed in the presence of 17 pg UNG, and samples C and D being performed in an absence of UNG. Samples E-H were performed utilizing 2.5 pmol ricin oligonucleotide substrate (SEQ ID NO.:4) with samples E and F being performed in the presence of 17 pg UNG and samples G and H being performed in an absence of UNG.

FIG. 14 illustrates an initial stage in a reaction performed in accordance with an alternative aspect of the present invention with Panel A illustrating the reaction state in an absence of N-glycosylase and Panel B illustrating the corresponding stage performed in the presence of N-glycosylase.

FIG. 15, Panels A and B illustrate a reaction event subsequent to that depicted in FIG. 14, Panels A and B, respectively.

FIG. 16, Panels A and B illustrate reaction events subsequent to those depicted in FIGS. 15, Panels A and B, respectively.

FIG. 17, Panels A and B illustrate reaction events subsequent to those depicted in FIG. 16, Panels A and B, respectively.

FIG. 18, Panels A and B, illustrate reaction events subsequent to those depicted in FIG. 17, Panels A and B, respectively.

FIG. 19, Panels A and B illustrate reaction events subsequent to those depicted in FIG. 18, panels A and B, respectively.

FIG. 20 illustrates the results of fluorescent analysis of control reaction utilizing product mimics. Samples A and B illustrate fluorescence produced by samples containing a sequence (SEQ ID NO.: 5) partially complementary relative to the oligonucleotide probe. Samples C and D illustrate fluorescence produced utilizing an oligonucleotide (SEQ ID NO.: 6) having fully complementary sequence relative to the oligonucleotide probe. Samples E and F illustrate the fluorescence resulting where no complementary oligonucleotide is present.

FIG. 21 illustrates fluorescent analysis results of control reaction utilizing product mimics in the presence (samples E-G) of Taq polymerase and absence (samples A-D) of Taq polymerase; and in the presence (samples C-G) of endonuclease Nb.BbvC I and absence of Nb.BbvC I (samples A and B). Samples A-F are performed in the presence of an oligonucleotide having sequence as set forth in SEQ ID NO.:7. Sample G is performed in an absence of product simulating nucleotide.

FIG. 22 illustrates the results of fluorescent analysis of test isothermal amplification reactions using product mimics. Samples A and H are performed in an absence of substrate and product. The ratio of substrate to product is varied to represent differing percentages as indicated along the x-axis. The substrate utilized had sequence as set forth in SEQ ID NO.: 12 with the product mimic having sequence as set forth in SEQ ID NO.: 13. Primer present in the reaction had sequence as set forth in SEQ ID NO. 14 and the intact probe had sequence as set forth in SEQ ID NO.: 15.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

In general the invention pertains to N-glycosylase detection methods and assays and includes assay components and assay kits. The general concepts and methodology of the invention as described herein can be utilized for N-glycosylases in general or can be utilized or adapted for specific detection of a particular glycosylase. Although toxins may often transform RNA substrates more rapidly than DNA substrates, only the latter are resistant to ubiquitous RNases that would readily degrade RNA substrates. Since the assays presented herein utilize DNA oligonucleotides, the term DNA N-glycosylase—rather than RNA N-glycosidase—will be utilized predominantly hereafter as appropriate. Although the assays and methods are described primarily with respect to specific classes and examples of glycosylases, as will be understood by those of ordinary skill in the art, the invention can be utilized for alternative specific glycosylases and alternative classes of glycosylases which are not specifically described herein.

Exemplary glycosylases involved in DNA repair for which the invention can be directly applied or can be adapted to detect or assess activity include but are not limited to: Endonuclease III (Nth), Endonuclease V, Endonuclease VIII, Fpg (New England Biolabs, Beverly Mass.), and Escherichia coli MutY. Exemplary DNA N-glycosylases that inactivate ribosomes due to RNA N-glycosidase activities for which the invention can be utilized include bryodins, dianthin 30, gelonin, luffin a, mapalmin, momordin I, pokeweed antiviral proteins, saporins, trichosanthin, trichokirin, abrin, ricin, ricin A-chain, Ricinus communes agglutinin 120, viscumin, and volkensin.

More specifically, the N-glycosylase methods and activity assays developed and presented herein are designed primarily to detect N-glycosylases which can be utilized therapeutically and/or as toxic agents. Typically, these DNA N-glycosylases will also be RNA glycosidases which depurinate ribosomal RNA. Many of such RNA glycosidases are adenine-specific and accordingly, the invention will be described primarily with respect to such adenine-specific glycosylases and glycosylase activity. It is to be understood however that the invention encompasses additional, specific and non-specific glycosylases.

In conventional N-glycosylase assays polynucleotide substrates have been utilized to detect or determine N-glycosylase activities. However, such assays can be particularly disadvantageous due to use of radio isotopes and/or the need for post-reaction separation techniques or derivatization of products that preclude rapid analysis. Many such conventional assays utilize substrates that are too large and/or undefined to permit toxin identification and are often subject to false positive signals due to contaminating or non-specific nucleases. In contrast, the present invention utilizes relatively small substrates in conjunction with one or more signal amplification reaction and a resulting fluorescent signal. As described more fully below, the methodology of the invention can be performed quickly, yield reliable and accurate results, and can be performed without post-reaction separation. Accordingly, the assays can be particularly useful for field deployment.

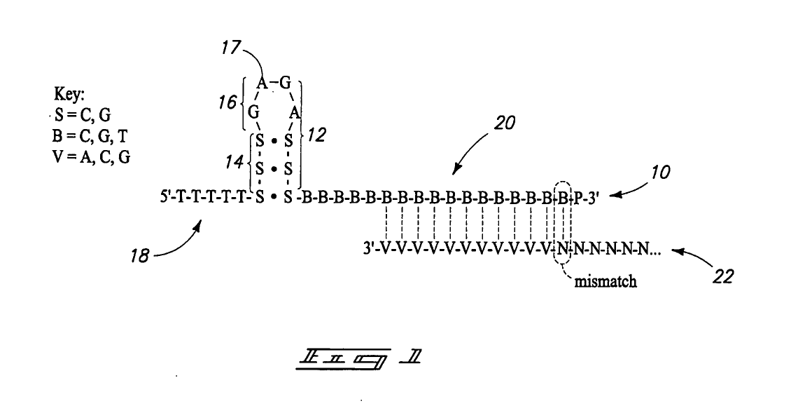

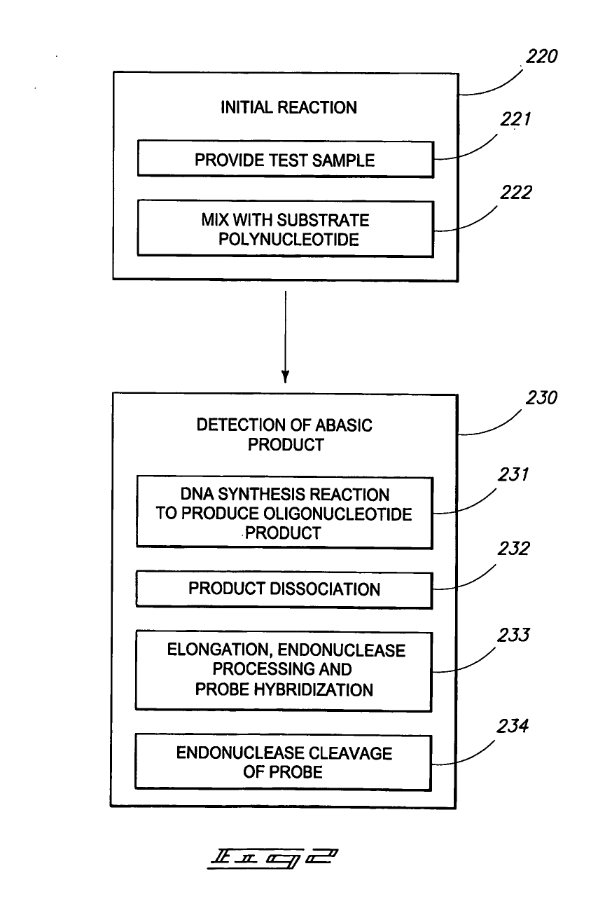

Referring to FIG. 1, the present invention uses small polynucleic acid substrates, typically oligomers of less than 50 bases. These oligonucleotides (oligos) are small enough to allow specific design to differentiate between toxins that remove adenine residues in different sequences from a given substrate or to differentiate between toxins from non-specific nucleases. An exemplary polynucleotide substrate 10 is shown in FIG. 1 which is designed for utilization for detection and/or assessment of N-glycosylase enzyme activity where the glycosylase has ricin-like activity. The general sequence of substrate 10 can be the sequence set forth in SEQ ID NO.:1. In general, the polynucleotide substrate can be a synthetic molecule having an ability to form an intramolecular hairpin loop 12. The hairpin loop shown in FIG. 1 is a ten-mer having a stem portion 14 comprising a two complementary sequences separated by a tetraloop 16 where the term “tetraloop” refers to a four-base looped sequence as illustrated.

Where ricin or a glycosylase having a ricin recognition site is to be tested for the tetraloop can have a ‘G-A-G-A’ sequence as shown. For the substrate illustrated in FIG. 1, a ricin depurination site 17 is comprised by the tetraloop of the hairpin structure. For alternative glycosylase enzymes the synthetic or artificial substrate can have a differing tetraloop sequence, can differ in stem length and/or can lack the hybridized stem portion all together. Regardless of whether a hairpin structure is present in the substrate, the artificial substrate can typically comprise s a 5′ polynucleotide sequence 18 comprising five or more bases disposed 5′ relative to depurination site 17. The synthetic substrate can additionally preferably comprise a 3′ sequence 20 disposed 3′ relative to the depurination site. The 5′ portion 18 can comprise the five-‘T’ sequence indicated, or comprise alternative sequence based upon site preference of the particular glycosylase. 3′ sequence 20 can comprise the 17-‘B’ sequence illustrated (where B═C, G, or T), or can comprise fewer or additional bases.

A particular sequence of nucleotides can be chosen for 3′ portion 20 based upon a particular sequence of a designed synthetic reverse primer 22. Typically, substrate 10 will comprise DNA and primer 22 will be designed to hybridize to a portion of 3′ region 20 and act as a reverse primer during synthesis of a nascent strand complementary to substrate 10. In accordance with one aspect of the invention, the sequence comprised by a portion of the 3′-region 20 of substrate 10 and its complementary portion of primer 22 are strategically developed to have a role in detection of N-glycosylase activity as further discussed below.

Reverse primer 22 can comprise a 3′ portion having the sequence illustrated in FIG. 1 and as set forth in SEQ ID NO.:2. As illustrated, the eleven nucleotides disposed at the 3′-terminus of the primer can align with nucleotides comprised by the 3′ portion 20 of the substrate, and preferably the eleven 3′ nucleotides of the primer are complementary to eleven consecutive nucleotides within the substrate. It can be additionally preferred that the most 3′ nucleotide of the substrate (position 32 of SEQ ID NO.:1) be non-complementary relative to the nucleotide at position 12 of the primer as counted from the 3′ end (corresponding to position 6 of SEQ ID NO.:2). Additionally, it can be preferred that the 3′-end of the substrate be “blocked” with a phosphate group in order to inhibit or prevent unwanted extension by Taq polymerase. Methodology and assays of the invention are developed based upon the ability of N-glycosylase to produce an abasic site in a substrate (such as depurination at target site 17 of exemplary substrate 10), in conjunction with differing products resulting from elongation of primer 22 as a function of whether the substrate is intact or has the abasic site.

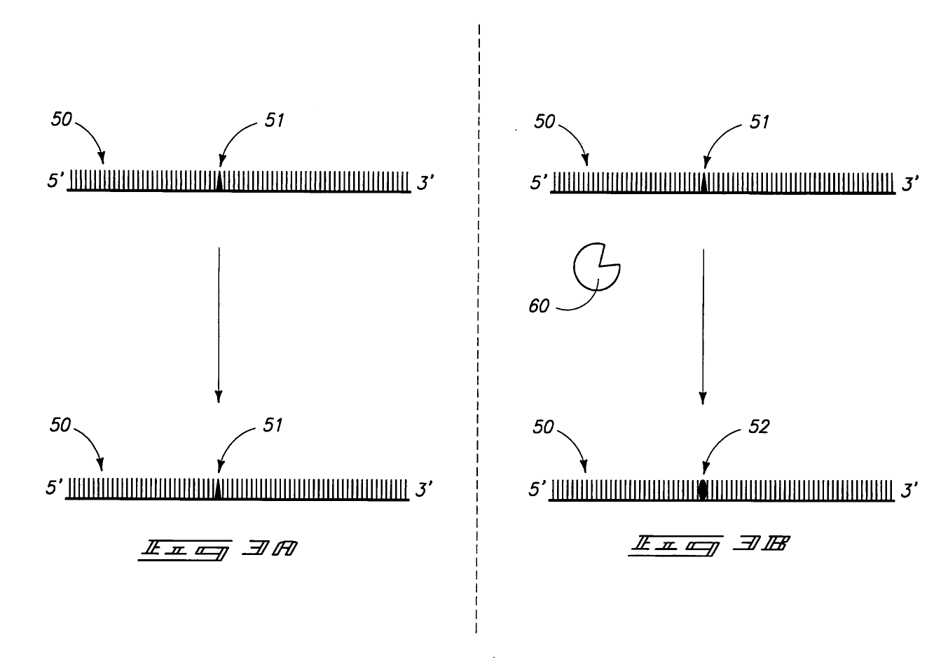

Referring to FIG. 2, such shows a flowchart generally illustrating methods and assay processes in accordance with the invention. As illustrated, the basic overall reaction scheme involves an initial reaction 220 followed by a detection process 230. In the initial reaction, a sample to be tested for the presence of N-glycosylase activity is provided in a preliminary process 221. Such test sample can be obtained and/or prepared for general or specific N-glycosylase detection, and/or to test the activity of a sample known to contain one or more N-glycosylases. The test sample is mixed with a substrate polynucleotide in stage 222. The substrate polynucleotide can preferably have a defined or known site for N-glycosylase base removal. Such substrate polynucleotide can be, for example, a substrate polynucleotide as depicted in FIG. 1, and as set forth in SEQ ID NO.:1. In initial reaction 220, where N-glycosylase is present in the test sample the glycosylase can convert the substrate polynucleotide to an abasic product. Typically, a single molecule of N-glycosylase can convert multiple molecules of substrate to the abasic product. Where the test sample lacks N-glycosylase, no abasic product is produced in the initial reaction.

Following the initial reaction, detection process 230 can be performed to detect abasic product formed in the initial reaction. Although the detection reaction can be conducted in a separate vessel, the detection process will typically be performed in the same vessel utilized for the initial reaction. Accordingly, the entire assay process of the invention can be performed in a single assay container.

As discussed more fully below the detection process 230 can be performed in a single stage or can be divided into two sequential stages. Regardless of whether the detection is performed in one or two stages, the detection can comprise the four processing events illustrated in FIG. 2. In an initial event, DNA synthesis conditions are produced where the substrate polynucleotide (either intact or abasic product form) is utilized as a template during DNA amplification reactions to produce an oligonucleotide product which is complementary to a portion or an entirety of the substrate polynucleotide sequence. The DNA synthesis reaction can typically be conducted by providing free nucleotides, an appropriate polymerase, such as a thermostable DNA polymerase (that pauses at abasic sites), and an appropriate reverse primer (discussed below). In general, the resulting oligonucleotide product length will be determined by the presence or absence of an abasic site produced during the initial reaction.

An exemplary nuclease which can be utilized for DNA amplification in accordance with the invention is Taq DNA polymerase which is a thermo-stable DNA polymerase originally isolated from Thermus Aquaticus. Taq polymerase is a 5′ to 3′ polymerase which is known to pause at abasic sites present in a template strand. Accordingly, utilization of Taq polymerase in the assays of the invention allows differentiation between abasic and intact polynucleotides with resulting products having differing lengths. The oligonucleotide product produced from a template having an abasic site will be shorter than the intact substrate polynucleotide and shorter than oligonucleotide products from extended-upon intact substrate-template molecules.

A product dissociation event 232 is included in the detection processing. In particular implementations of the invention, product dissociation is accomplished utilizing application of heat to the reaction to melt or dissociate the oligonucleotide product from the template strand. In particular instances, thermo-cycling (repetitive heating and cooling) can be conducted to achieve multiple rounds of DNA amplification and product dissociation. Dissociation of the product oligonucleotide can allow a single template molecule to be utilized for production of multiple oligonucleotide products. Accordingly, a single abasic substrate polynucleotide molecule produced in the initial reaction 220 can be amplified by production of multiple oligonucleotide products which are short relative to products produced from a full length intact substrate molecule. Accordingly, the initial “signal” abasic site can be amplified by production of multiple shortened products.

In alternative implementations of the invention, DNA synthesis/amplification is conducted at a temperature sufficiently low to allow hybridization of oligonucleotides in an absence of thermal cycling. Endonuclease processing nicks oligonucleotide products extended by Taq polymerase such that shortened oligonucleotides can dissociate from template oligonucleotides immediately upon their formation without a change in temperature. This processing can allow amplification of the original abasic signal without utilizing thermocycle equipment.

The dissociated product is subjected to elongation and probe hybridization in a subsequent processing event 233. As will be discussed in more detail below, substrate polynucleotides utilized in the initial reaction 220 are specifically designed such that oligonucleotide products produced from an abasic template are able to undergo elongation after product dissociation and hybridization to a new template; while product oligonucleotides produced using intact substrate polynucleotides do not undergo further elongation. In particular implementations of the invention, hybridization of an extended (and then dissociated) oligonucleotide product onto a new template molecule allows or promotes further elongation. In an alternative implementation, hybridization of an extended (then dissociated) oligonucleotide product upon itself allows further elongation of the resulting hairpin stem.

Probes utilized for the invention typically contain an oligonucleotide at least a portion of which is complementary to a sequence of the oligonucleotide product produced in the DNA amplification processing 231. Typically, the probe will have a probe oligonucleotide sequence containing from about 14-40 nucleotides.

Probes in accordance with the invention are typically dual labeled and in particular instances have a fluorescent label and a corresponding fluorescent quencher moiety. A fluorescent label can be proximate or preferably directly on a first end of the probe oligonucleotide, with the quencher label being proximate or directly on the opposing end. The invention contemplates utilizing the fluorescent label covalently linked either proximate the 5′ or the 3′ end with the corresponding quencher moiety being covalently linked proximate the opposing end.

Numerous fluorescent labels and fluorophore/quencher pairs are available for utilization for labeling oligonucleotide probes in accordance with the invention. An exemplary fluorophore which can be utilized is fluorescein (FAM). Such fluorophore can be utilized with a “black hole quencher” label such as black hole quencher 1 (BHQ1). It is to be understood that the invention contemplates utilization of alternative fluorophore quencher pairs.

In accordance with the invention, detection probes can comprise specifically designed oligonucleotide sequences which comprise an endonuclease cleavage site. As illustrated in FIG. 2, an endonuclease cleavage step 234 can result in endonuclease cleavage of the probe at the endonuclease cleavage site. Preferably the probe is designed such that hybridization of the probe oligonucleotide to the complementary sequence present in the DNA amplification product (prior to or after elongation) creates a duplex DNA recognition site which is recognized by an endonuclease. Such probe oligonucleotide is preferably designed such that the probe is cleaved by the endonuclease to produce independent probe fragments thereby decreasing fluorescent quenching to produce a fluorescent signal. Preferably, repeated rounds of probe hybridization and endonuclease cleavage results in further signal amplification. Such multiple amplifications (amplification cascade) allow methodology and assays of the invention to be extremely sensitive and capable of detecting minute quantities of N-glycosylase in a test sample.

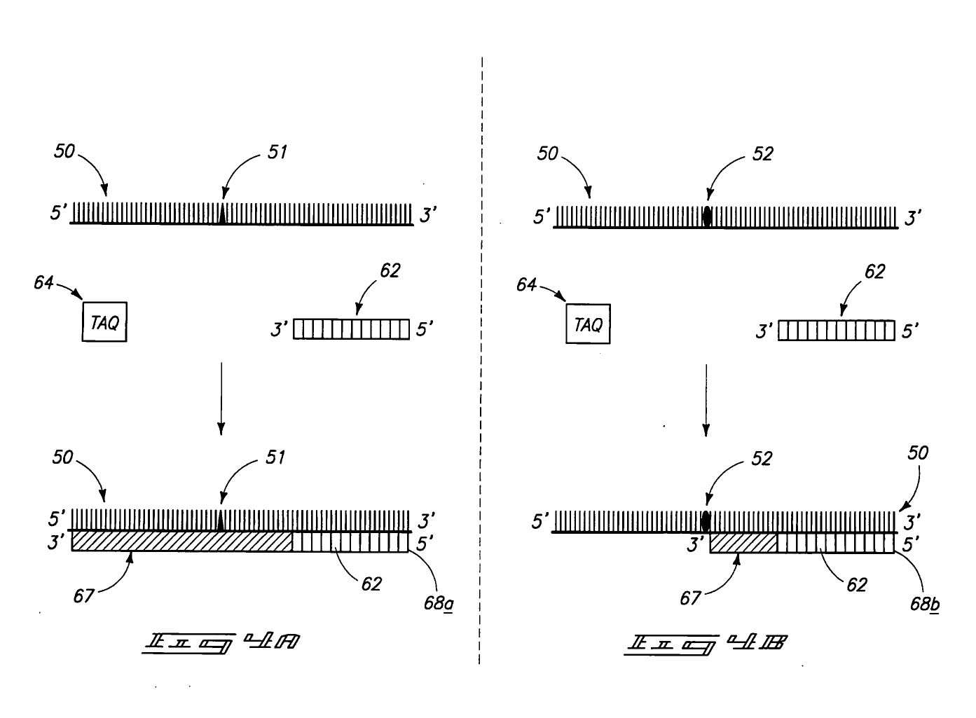

Two distinct exemplary implementations of methodology of the invention are described below. A first implementation which can be referred to as a 3-level cascade signal amplification assay is described with reference to FIGS. 3-8. It is to be understood that the schematic representations shown in the figures are for description purposes and are not drawn to scale or intended to reflect relative sizes of molecules represented therein. Each of FIGS. 3-8 includes a first part (Panel A) showing a process in an absence of N-glycosylase and a second part (Panel B) representing events where N-glycosylase is present in a test sample.

Referring to FIG. 3 Panel A, a substrate polynucleotide 50 is illustrated having a particular base removal site 51. As described above, substrate 50 and base removal site 51 can be specifically designed and synthesized to detect general N-glycosylase activity or can be designed and synthesized to be specific for a particular glycosylase enzyme. Where the N-glycosylase to be detected is ricin or has ricin-like recognition and base removal properties, substrate 50 can be designed to be capable of forming an intra-molecular hairpin loop as described above. Regardless of the specific design, the substrate polynucleotide preferably consists of a single strand DNA molecule having at least one base capable of removal by glycosylase activity to produce an abasic site. Preferably the abasic site will be produced at a known position within the substrate sequence. In particular implementations of the invention, substrate 50 can comprise a sequence as set forth in SEQ ID NO.: 1 and as illustrated in FIG. 1.

As shown in FIG. 3 Panel A, substrate 50 remains intact in the absence of N-glycosylase due to non-removal of the base at target site 51. Referring to FIG. 3 Panel B, the base at target site 51 is removed in the presence of a glycosylase 60 to produce an abasic site 52. It is to be noted that a single N-glycosylase molecule 60 can remove a base from multiple substrate polynucleotide molecules. The resulting abasic substrate molecules can be referred to as an initial signal in the assay process.

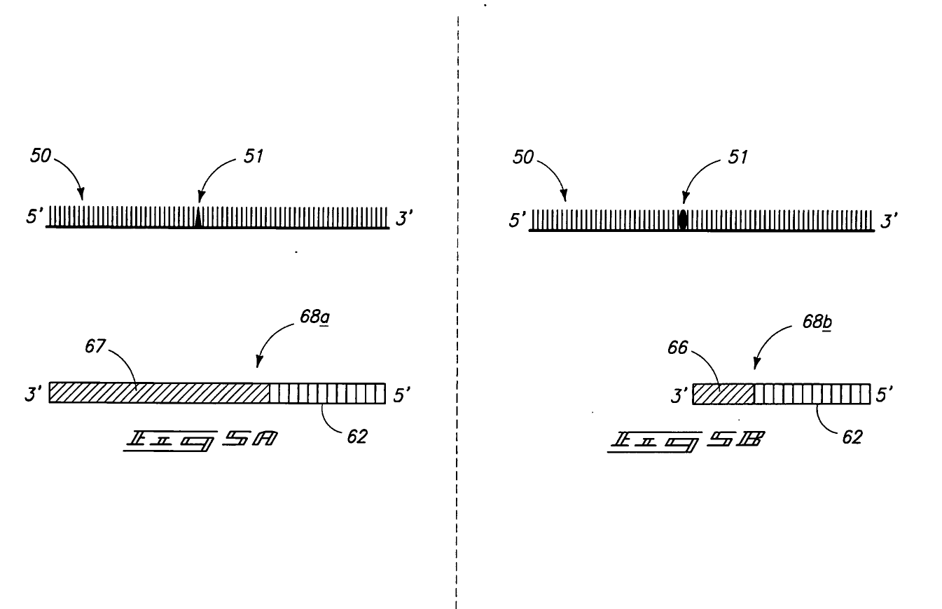

Referring to FIG. 4, such illustrates an amplification reaction where substrate 50, either intact (Panel A) or having an abasic site (Panel B), is utilized as a template during DNA synthesis in a DNA amplification reaction.

The amplification process shown in FIG. 4 is conducted subsequent to the N-glycosylase reaction shown in FIG. 3. However, the DNA amplification reaction can be conducted in the same reaction vessel as the N-glycosylase reaction by providing an appropriate polymerase reaction buffer, as will be understood by those of ordinary skill in the art, and providing a polymerase primer 62 and an appropriate polymerase 64.

Primer 62 is preferably of sufficient length to promote polymerase activity and extension of the primer as depicted in FIGS. 4 Panels A and B, bottom. The sequence of primer 62 is not limited to a particular sequence and preferably comprises a sequence portion which is complementary to a portion of the substrate 50 sequence proximate the substrate 3′ end to allow sufficient hybridization to serve as a primer. It is noted that the 5′ end of primer 62 is shown to correspond directly to the 3′ end of the corresponding substrate in FIGS. 4A and B. However, the invention contemplates primers which extend beyond the 3′ end of the substrate (as depicted in FIG. 1). Primer 62 preferably has one or more nucleotides that are mismatched relative to the extreme 3′ terminus of the substrate molecule to inhibit or prevent unwanted extension of the substrate with the primer acting as template. Additionally, substrate molecule 50 can preferably contain a 3′-phosphate group to redundantly block such unwanted extension.

Primer 62 can comprise a sequence as set forth in SEQ ID NO.: 2, especially where the substrate has sequence as set forth in SEQ ID NO.: 1. However, as described above, alternate substrate sequence can be utilized and accordingly sequence of oligonucleotide primer 62 can vary.

Referring to FIG. 4 Panel A, where an intact substrate is utilized (lacking an abasic site), hybridization of primer 62 and polymerase activity can extend the primer to produce a long extension portion 67 and form a “full length” nascent extension product 68a. In contrast, referring to FIG. 4 Panel B, extension of hybridized primer 62 by, for example, Taq polymerase (which pauses at abasic site 52) results in a short extension portion 66. The overall extension product 68b produced from the abasic template is short relative to the full length extension product 68a depicted in Panel A.

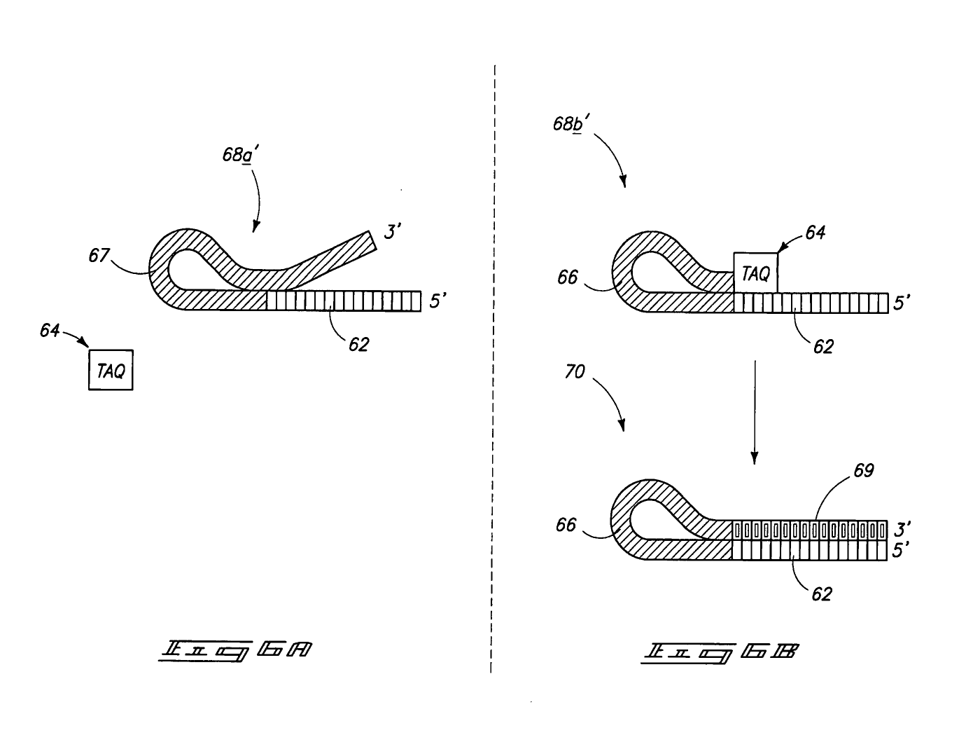

Referring to FIG. 5 Panels A and B, the respective extension products 68a and 68b are dissociated from template strands 50 to produce single stranded extension products. Such dissociation can be accomplished by “melting” the DNA by subjecting the reaction mixture to a temperature increase. In the exemplary implementation of the assay as depicted in FIGS. 3-8 (the 3-level cascade mechanism) the DNA amplification method in FIGS. 4 and 5 are preferably performed using cyclic heating and cooling, or “thermocycling”. Accordingly, by providing excess nucleotides and excess primer 62, multiple extension products can be formed from each template polynucleotide. This amplification can thereby increase or amplify the initial signal by producing multiple shortened extension products 68b for each abasic substrate in the reaction.

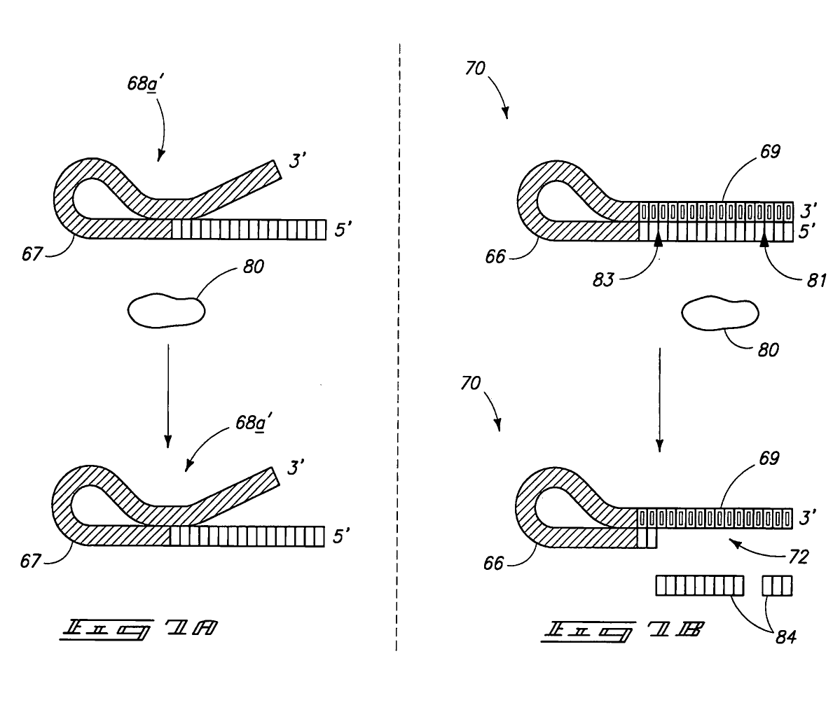

Referring next to FIGS. 6 Panels A and B, it is noted that the full length extension product 68a comprises complementary portions allowing the extension product to form an intramolecular hairpin loop structure 68a′. This intermolecular hybridization occurs upon cooling after dissociation of the extension product. Since the full length extension product comprises a 3′ terminal sequence portion that is non-complementary relative to the primer portion 62, hybridization does not occur in the terminal portions of the molecule. Accordingly, polymerase 64 does not further elongate the extension product. With reference to FIG. 6 Panel B, the shortened extension product, which also contains complementary portions, is able to form a hairpin type structure 68b′ upon cooling. However, because of the presence of a shorter extension region 66 the extension portion is able to act as a primer for extension of the molecule by the polymerase. An additional extension portion 69 is produced which is complementary to primer 62 resulting in a further extended molecule 70. The further elongation of intramolecularly hybridized molecule 68b′ can occur during the amplification reactions shown in FIGS. 4 and 5.

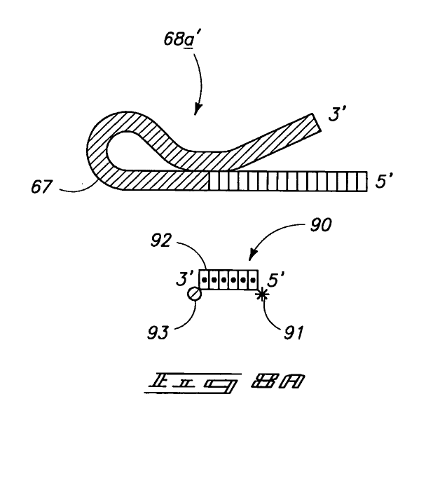

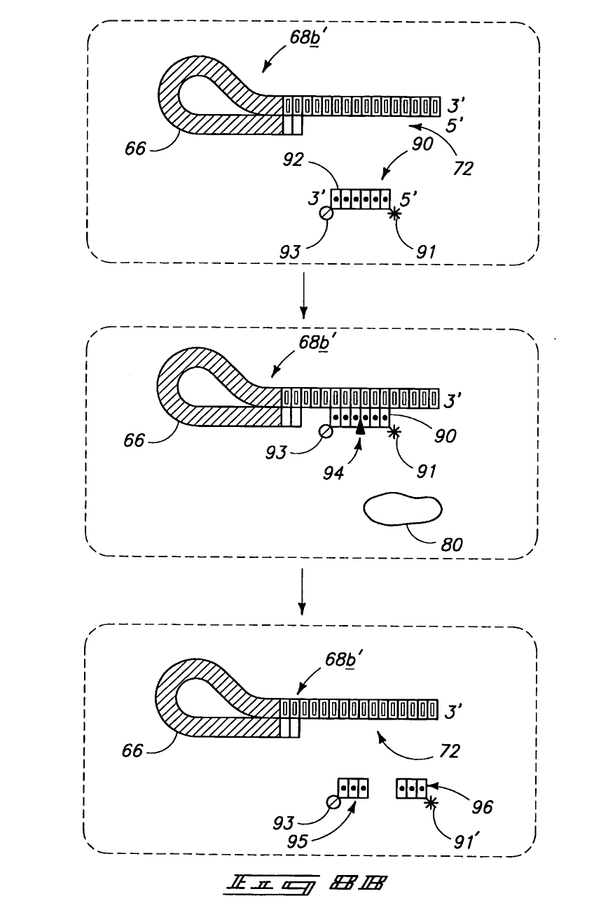

Upon completion of the thermocycling during amplification reactions, additional processing events can be conducted as depicted in FIGS. 7 and 8. In the particular implementation illustrated an endonuclease or “nicking” enzyme 80 is added to the assay mixture subsequent to completion of thermocycling. Nicking enzyme 80 can preferably be a site and strand specific endonuclease that cleaves only one strand of DNA within its recognition sequence on a double-stranded DNA substrate. Preferably, primer 62 contains a sequence having one or more nicking sites 81 and 83 as shown on panel 7B, top. Formation of the additional extension portion 69 in the subsequent process forms a sequence complementary to the 5′-portion of the previously extended molecule 70, which corresponds to the 5′-portion of primer 62. The primer is specifically designed such that formation of the additional extension 69 completes the recognition site(s) for recognition by endonuclease 80 and nicking at cleavage sites 81 and 83. As illustrated at the bottom of FIG. 7 Panel B, nicking of the primer sequence produces primer fragments 84 which can dissociate from extended molecule 70 leaving a single strand 3′ terminus region 72. As shown in FIG. 7 Panel A, since the 5′ and 3′ ends of the full length extension product are non-complementary, the recognition sequence for endonuclease 80 is not created in the intramolecularly hybridized form 68a′ of the molecule and nicking does not occur.

Referring now to FIG. 8, a detection probe 90 is provided into the assay mixture. Referring to panel 8B, detection probe 90 has an oligonucleotide portion 92 labeled with a first label 91 and a second label 93. Probe 90 can be a detection probe as described above. The sequence of oligonucleotide portion 92 can preferably be complementary to at least a portion of the additional extension region 69. Due to nicking of the primer and dissociation of primer fragments, probe 90 can hybridize to the single stranded portion of the intramolecularly hybridized molecule 68b′ as depicted in the second portion of Panel B. Probe 90 preferably has a nicking site 94 similar to the primer nicking site described earlier. Upon hybridization of the probe, endonuclease 80 is able to recognize the duplex DNA recognition site and cleave the probe at site 94 producing independent probe fragments 95 and 96. Such probe fragments are able to dissociate from the intramolecularly hybridized molecule 68b′ as illustrated in the bottom of Panel B.

Where the probe labels comprise a fluorophore and quencher, fragmentation of the probe decreases or eliminates quenching thereby increasing fluorescence. Upon dissociation of the fragmented probe an additional probe molecule can hybridize to molecule 68b′ and can be cleaved by the endonuclease 80. Repetitive rounds of probe hybridization and fragmentation can result in further amplification of the signal increasing assay sensitivity without temperature cycling.

Referring to FIG. 8 Panel A, as illustrated probe 90 does not have a corresponding complementary sequence on full length hybridized molecule 68a′ and therefore does not bind to the full length elongation product. Since no recognition site is created probe 90 remains intact and the fluorescence remains quenched in the assay samples in an absence of N-glycosylase activity.

In the “3-level cascade signal amplification” assay described above, the first level can be described as the toxin or N-glycosylase reaction. The second level, which is performed subsequently to the first level, is the DNA amplification process. The third level is performed subsequent to the DNA amplification and involves endonuclease activity to cleave both primer and probe oligonucleotides. Presented below are examples and control reactions for the described 3-level cascade signal amplification assay described.

N-glycosylase reactions were conducted utilizing polymerase chain reaction. (PCR) tubes in 5 μl volumes at a temperature of 30° C. to 37° C. A ricin substrate synthetic oligonucleotide was designed having the sequence set forth in SEQ ID NO.:4. However, for test reactions uracil N-glycosylase was utilized (UNG) as surrogate and a UNG substrate was synthesized having the sequence set forth in SEQ ID NO.:3. It is noted that SEQ ID NOS.: 3 and 4 are identical other than a single substitution of uracil and position 9 in place of adenine. It is further noted that the nucleotide at position 9 in each of SEQ ID NOS.:3 and 4 is the site of base removal. 2.5 pmol of polynucleotide substrate was utilized in the N-glycosylase reactions. The reaction was conducted in an appropriate buffer (for UNG reactions 10 mM Tris, 1 mM EDTA at pH 8.0). It is noted that ricin assays are appropriately conducted utilizing AKT buffer (7 mM Na acetate, 100 mM KCl, 0.1% (v/v) Triton X-100, pH 4.0). Reactions were conducted for approximately 5 minutes or longer and were stopped by incubation at 94° C. for 2 minutes.

In the second level of the assay, 50 μl of amplification reagents are added in one step to the reaction tubes in which the N-glycosylase reactions were performed. The amplification reagent mixture contained, per 1000 μl: 850 μl nuclease-free water; 100 μl 10× NEB buffer #2; 20 μl dNTP mix (10 mM each of dGTP, dCTP, dATP, and dTTP); 20 μl Taq polymerase (5 units/μl); and 10 μl primer (100 pmol/μl). The primer utilized had the sequence as set forth in SEQ ID NO.:8. Upon addition of the amplification reagent mixture, and mixing of reaction tube contents, the reaction tubes were placed in a thermocycler. Typically, 10 thermocycles were utilized with a melting/extension temperature of 70° C. for 15 second and annealing/extension temperature of 47° C. for 30 seconds. Upon completion of thermocycling the temperature was decreased to 4° C.

In the third level of the assay, after completion of thermocycling 5.5 μl of nicking reagents were added in a single addition to the sample tubes in which the previous reactions had been conducted. The nicking reagent mixture contained, per 100 μl; 63 μl nuclease-free water; 10 μl 10× NEB buffer; 18 μl probe (200 pmol/μl); and 9 μl nicking endonuclease. The particular endonuclease utilized was Nb.BbvC I (10 units/μl). The probe utilized included a 5′ fluorescein label (FAM) and a 3′ black hole quencher 1 label (BHQ1) and had a probe oligonucleotide sequence as set forth in SEQ ID NO.:9. The nicking reactions were then incubated at approximately 37° C. for 15 minutes and subsequently at 94° C. for 2 minutes to stop the reactions. The reaction samples were then maintained at 4° C. until conducting fluorescence analysis. Fluorescence analysis was conducted on a UV light box with irradiation at 302 nm to obtain images as presented in the subsequent figures.

Control reactions were performed utilizing synthetic oligonucleotides that mimicked putative products formed as a result of N-glycosylase activity. A first of the mimic oligonucleotides was produced to mimic a substrate polynucleotide after base removal. This mimic oligonucleotide can be referred to as the abasic mimic. The sequence of the abasic mimic is set forth in SEQ ID NO.:10 where “n” is a stable (non-aldehyde) abasic site (based on tetrahydrofuran that does not undergo opening of the deoxyribose ring). Controls were also performed utilizing a second oligo-mimic based upon the 3′ portion of a product oligonucleotide that would remain if the product were hydrolyzed (cleaved abiotically) at the abasic site formed by the N-glycosylase. The sequence of the “cleaved product” oligonucleotide is set forth in SEQ ID NO.:11.

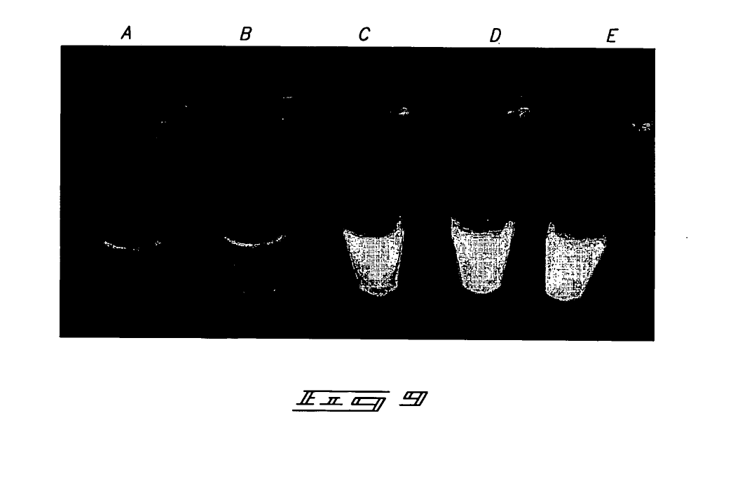

The control reactions were performed by adding Taq polymerase and amplification reagents to reaction tubes containing 2.5 pmol of a particular oligonucleotide(s). Twenty five thermocycles were conducted. After thermocycling, nicking endonuclease and probe (50 μl) were added to 5 μl of each reaction followed by incubation for 10 minutes at 47° C. The reactions were stopped by exposing to 94° C. temperature for 2 minutes. Sample A of FIG. 9 corresponds to a control utilizing no oligonucleotide. Sample B corresponds to the ricin substrate oligonucleotide (SEQ ID NO.:4). Sample C corresponds to a control performed utilizing the abasic mimic. Sample D corresponds to a control performed utilizing the cleaved product oligonucleotide (SEQ ID NO.: 11); and Sample E is a control performed utilizing synthetic nicked template having the nucleotide sequence set forth in SEQ ID No. 16. The synthetic nicked template functionally mimics oligonucleotide 70 in FIG. 7B after the double nicking by enzyme 80. However, the synthetic nicked mimic has a loop segment (in portion 66) that is eight bases shorter than the actual nicked template (SEQ ID No 17) would have.

Sample B containing the ricin substrate is indistinguishable from the tube containing no oligonucleotide substrate/product (sample A) with respect to fluorescence. This sample represents the background signal derived from the intact probe in solution.

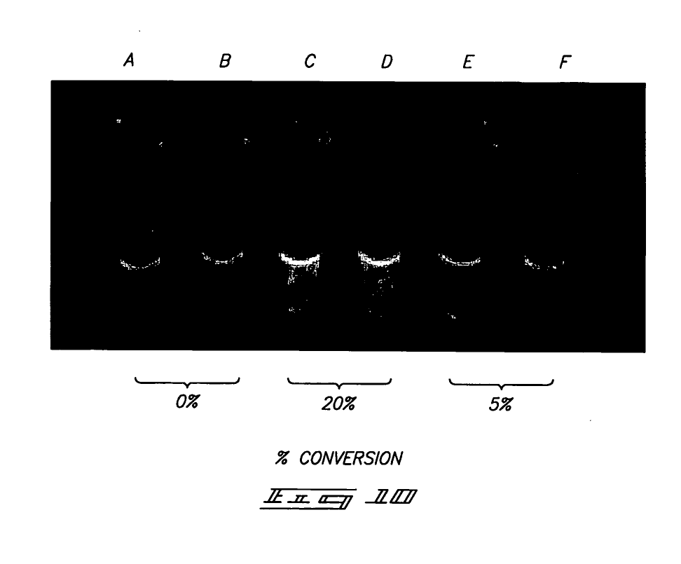

Referring to FIG. 10, control studies were additionally performed utilizing the procedure set forth for the controls presented in FIG. 9 with the exception that fewer (10) thermocycles were conducted. In these reactions, 2.5 pmol total oligonucleotide was utilized where the total is a combined amount of substrate oligonucleotide (SEQ ID NO.:4) and the abasic mimic oligonucleotide. These control reactions (performed in an absence of glycosylase) indicate the fluorescence level for various percent conversion of substrate to product. Samples A and B contain 2.5 pmol of substrate without synthetic product. Samples C and D contain 20% of the synthetic product (80% substrate). Samples E and F were performed utilizing 5% abasic mimic and 95% substrate polynucleotide (corresponding to 125 fmoles abasic mimic).

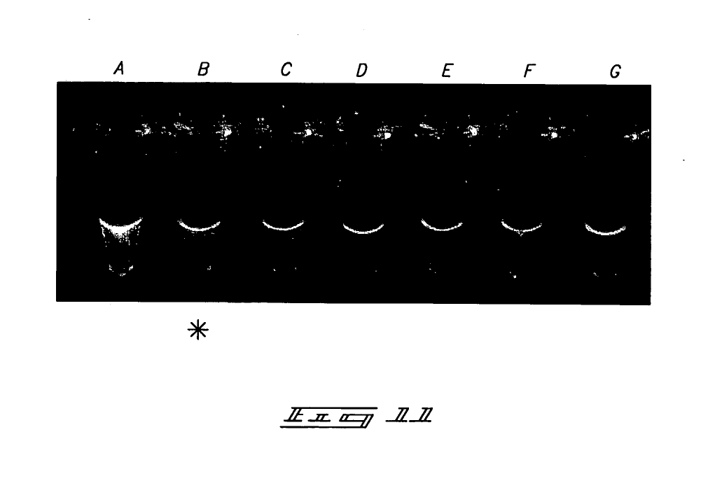

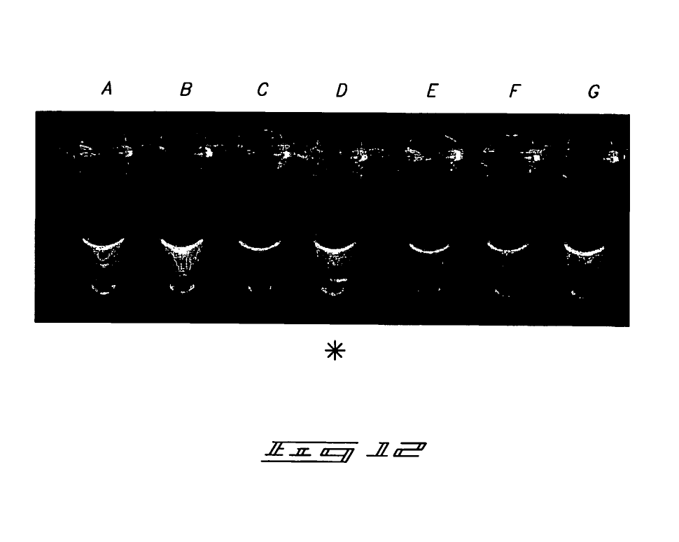

Additional N-glycosylase 3-level assays were performed to determine the sensitivity and detection limit of the assay. These studies utilized uracil N-glycosylase (UNG), and the UNG substrate polynucleotide (SEQ ID NO.: 3). 2.5 pmol of the substrate polynucleotide was utilized in each reaction with reaction samples containing differing amounts of uracil N-glycosylase enzyme. The results of such studies are presented in FIGS. 11 and 12.

Referring initially to FIG. 11, samples A-G were processed by performing the initial N-glycosylase reaction for 5 minutes at 25° C. Amplification was then conducted utilizing 10 thermocycles (47° C. for 30 seconds, 70° C. for 15 seconds). Nicking reactions were then conducted in the presence of probe at 37° C. for 15 minutes. Sample A contained 34 pg UNG. Sample B contained 3.4 pg UNG. Sample C contained no UNG. Sample D contained 340 fg UNG. Sample E contained 34 fg UNG. Sample F contained no UNG. Sample G represents a control containing 10% conversion equivalent (as described with respect to FIG. 10). For these particular processing times the detection limit is represented by sample B.

Referring to FIG. 12, a detection/sensitivity study was performed utilizing extended reaction times. The N-glycosylase reaction was conducted for 60 minutes at 25° C. DNA amplification was conducted utilizing 25 thermocycles (47° C. for 30 seconds, 70° C. for 15 seconds), and the third level nicking reaction was conducted at 37° C. for 15 minutes. Sample A contained 17 pg UNG. Sample B contained 1.7 pg UNG. Sample C contained an absence of UNG. Sample D contained 170 fg UNG. Sample E contained 17 fg UNG. Sample F contained no UNG. Sample G is a control containing 10% conversion equivalent. For this particular process study, the 170 fg UNG sample (sample D) is the detection limit. Such results indicate that sub-picogram quantities of glycosylase are detectible utilizing methodology of the invention.

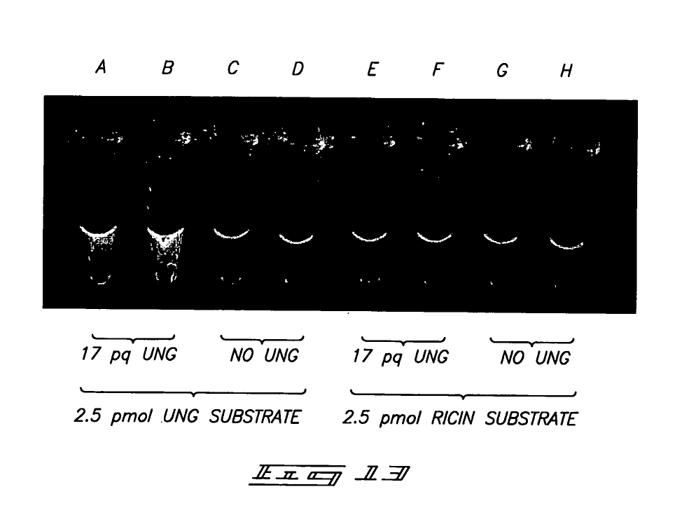

Referring to FIG. 13, additional study was performed to determine the specificity of uracil N-glycosylase for uracil-containing oligonucleotides. In samples A-D 2.5 pmol UNG substrate (SEQ ID NO.: 3) was utilized per reaction. In samples E-H 2.5 pmol ricin substrate (SEQ ID NO.: 4) was utilized per reaction. Samples A, B, E and F each contained 17 pg UNG while samples C, D, G and H were performed in an absence of UNG. Each sample was processed by performing an initial N-glycosylase reaction for 5 minutes at 30° C. followed by second level DNA amplification for 10 thermocycles (47° C. for 30 seconds, 70° C. for 15 seconds) followed by nicking reactions conducted at 37° C. for 15 minutes. These results indicate that the ricin oligonucleotide substrate (SEQ ID NO.: 4) does not undergo base removal by uracil N-glycosylase.

The 3-level cascade amplification methodology described above advantageously allows single-tube fieldable assays with high sensitivity. The assay can be combined with current antibody-based strategies for concentrating and purifying toxins from samples. Such assay is also complementary to antibody-based detection assays. Additionally, the completed assays (post-nicking reaction) can be stored at 4° C. prior to fluorescent analysis. Such samples can be stored stably at such temperature at least for hours and potentially for days.

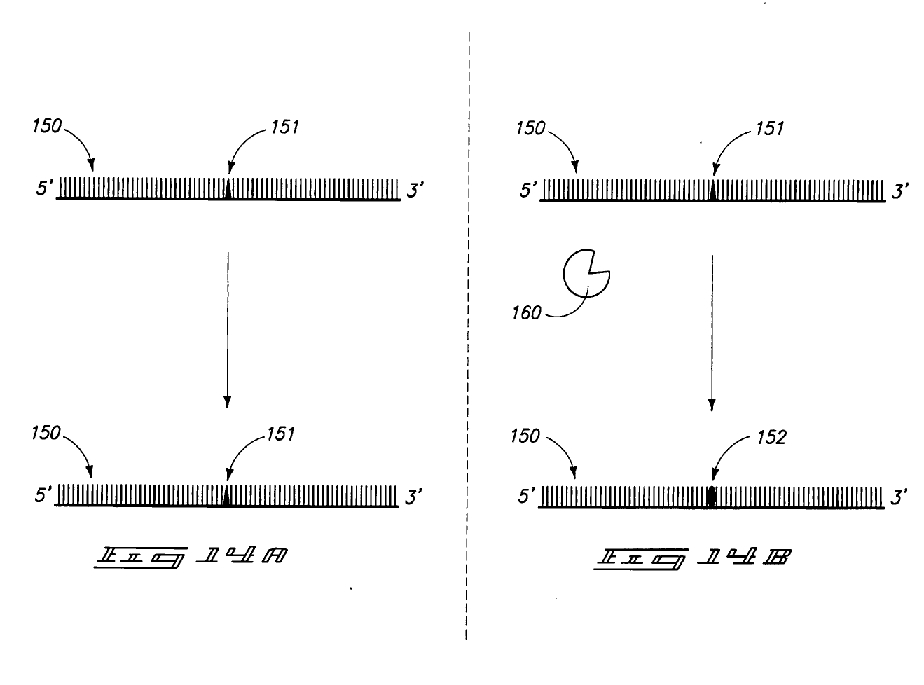

An alternative implementation of the invention utilizing a 2-step N-glycosylase assay method is described with reference to FIGS. 14-19. In such implementation, signal amplification is conducted isothermally (without thermocycling). The two-step assay involves a first step of forming an abasic product and a subsequent second step where reagents for polymerase reactions and endonuclease reactions are provided simultaneously rather than being run sequentially as discussed above with respect to the 3-level assay. Typically the detection amplification portion of the 2-step assay is conducted at 50° C. where Taq polymerase has about 10% of its maximum activity and where the exemplary endonuclease Nb.BbvC I can operate for a limited time.

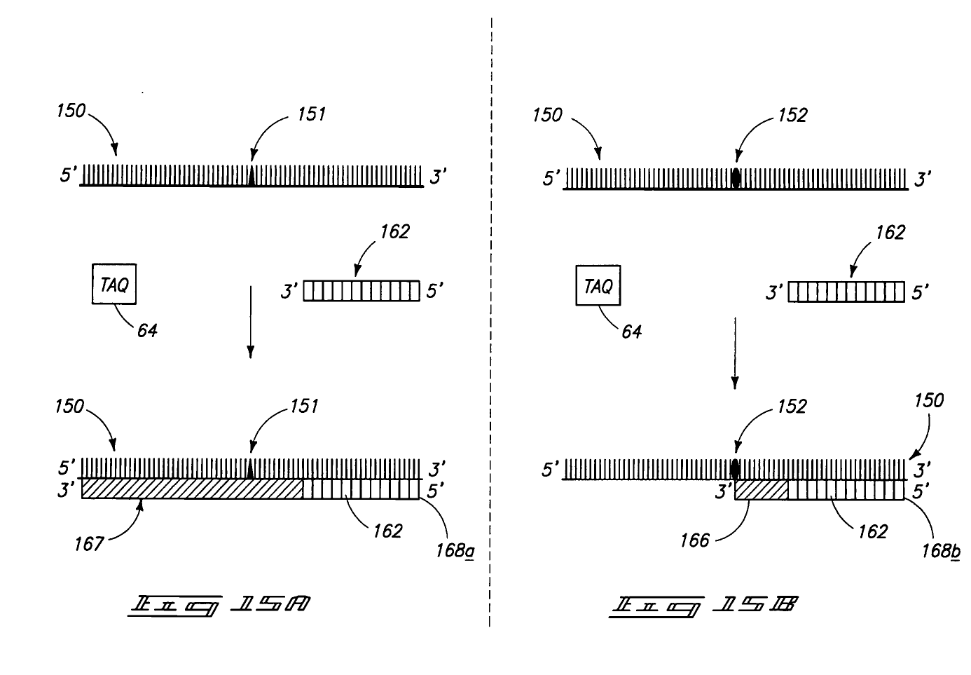

Referring to FIG. 14 Panels A and B it is again noted that panel A reflects events in an absence of glycosylase (or where the substrate does not undergo base removal) while panel B depicts events that occur in the presence of N-glycosylase which results in base removal. FIGS. 15-19 are also split to illustrate events in an absence of base removal (Panel A) and a presence of base removal (Panel B). As illustrated in FIG. 14 Panel A, in an absence of N-glycosylase substrate polynucleotide 150 remains intact retaining the base at target site 151. As shown in Panel B a glycosylase 160 can remove a base from target site 151 on substrate 150 to produce an abasic site 152 similar or identical to that described above with respect to the 3-level assay. Substrate 150 can comprise, for example, a sequence as set forth in SEQ ID NO.: 1. The particular sequence of the substrate can depend upon, for example, the particular N-glycosylase to be detected and to provide appropriate hybridization and endonuclease recognition sites as described below.

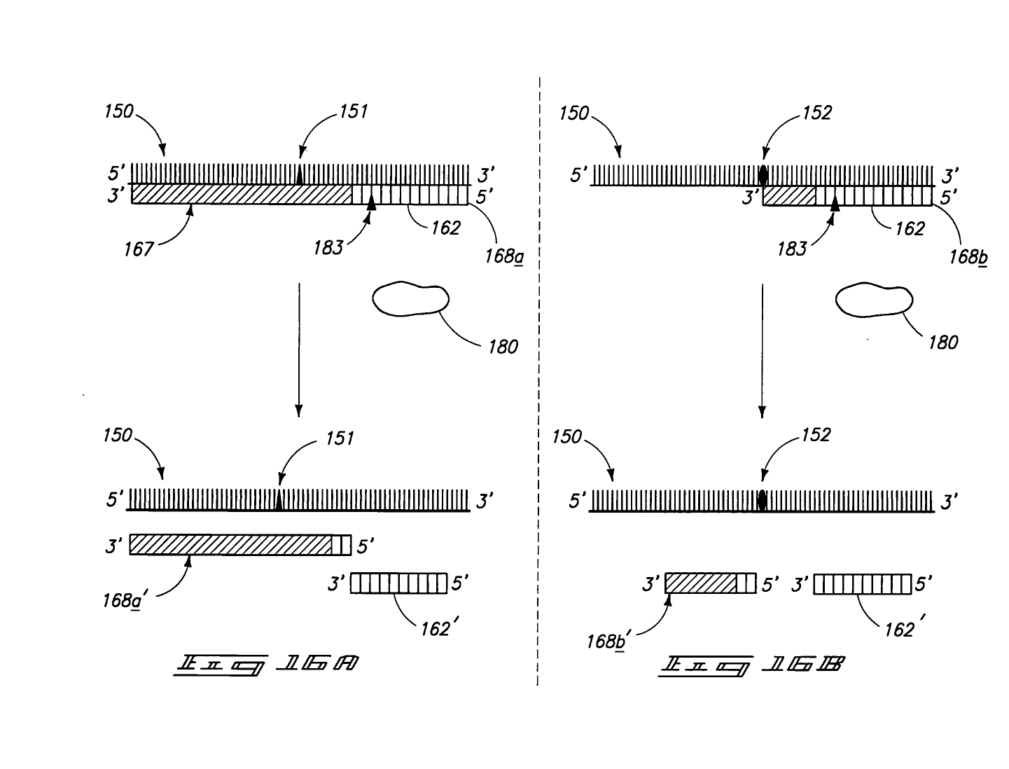

Referring to FIG. 15, as shown in Panel A, a primer 162 can be added. Primer 162 preferably comprises a sequence complementary to a sequence comprised at or near the 3′ end of substrate 150. The primer can preferably serve as an extension primer during a primer extension reaction in the presence of a polymerase 64 such as, for example, Taq polymerase. The extension of the intact substrate results in a full length extension molecule 168a by adding extension portion 167 which extends beyond the target site 151 of the template molecule. In contrast, with reference to FIG. 16 Panel B, hybridization of primer 162 can allow primer extension by Taq polymerase to yield a shortened extension portion 166 resulting in a shorter extension product 168b as a result of pausing of the polymerase at abasic site 152 on the template strand.

Referring to FIG. 16 an endonuclease 180 present in the reaction can be utilized to cleave primer 162 at a specifically designed nicking site 183. Preferably endonuclease 180 has a duplex-DNA recognition site and is site and strand specific to cleave only at site 183. As illustrated in Panel A, nicking of primer portion of extended molecule 168a allows dissociation of primer fragment 162′ and extended product fragment 168a′ from template strand 150. Referring to FIG. 16 Panel B, site specific cleavage at target site 183 of primer portion 162 of the shortened extended molecule 168b allows dissociation of primer fragment 162′ and a short extension product 168b′. It is to be noticed that in instances of primer cleavage and subsequent dissociation, a new primer molecule 162 can hybridize to the template strand containing abasic site to serve in an additional primer extension reaction thereby amplifying the initial signal.

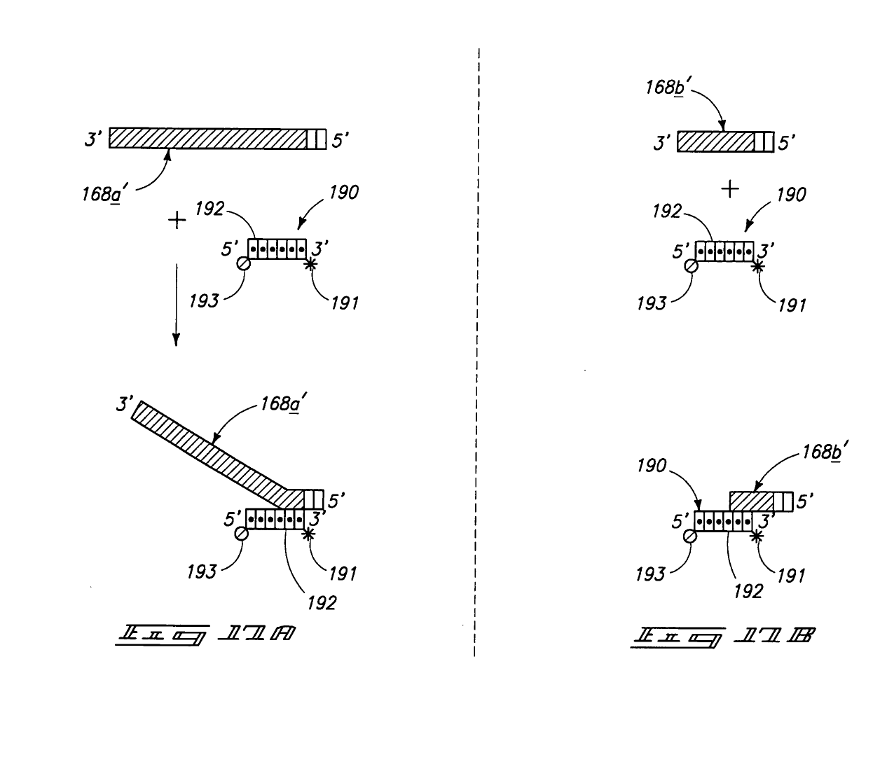

Referring next to FIG. 17 Panel A, a probe 190 is provided in the assay mixture and has an oligonucleotide portion 192 labeled with a first label 191 and a second label 193. Labels 191 and 193 can be, for example, probe labels as described above and in particular instances will be a fluorescent/quencher pair. Referring to the bottom of FIG. 17 Panel A the sequence of oligonucleotide 192 portion of probe 190 preferably contains a segment having a sequence complementary to a portion of extension fragment 168a′. This complementary sequence is preferably limited to a small portion of oligonucleotide sequence 190 leaving a second portion non-hybridized. Referring to FIG. 17 Panel B probe 190 is similarly able to hybridize to at least a portion of short extension product 168b′. As illustrated at the bottom of panel B a portion of the oligonucleotide 192 of probe 190 remains non-hybridized to extension fragment 168b′.

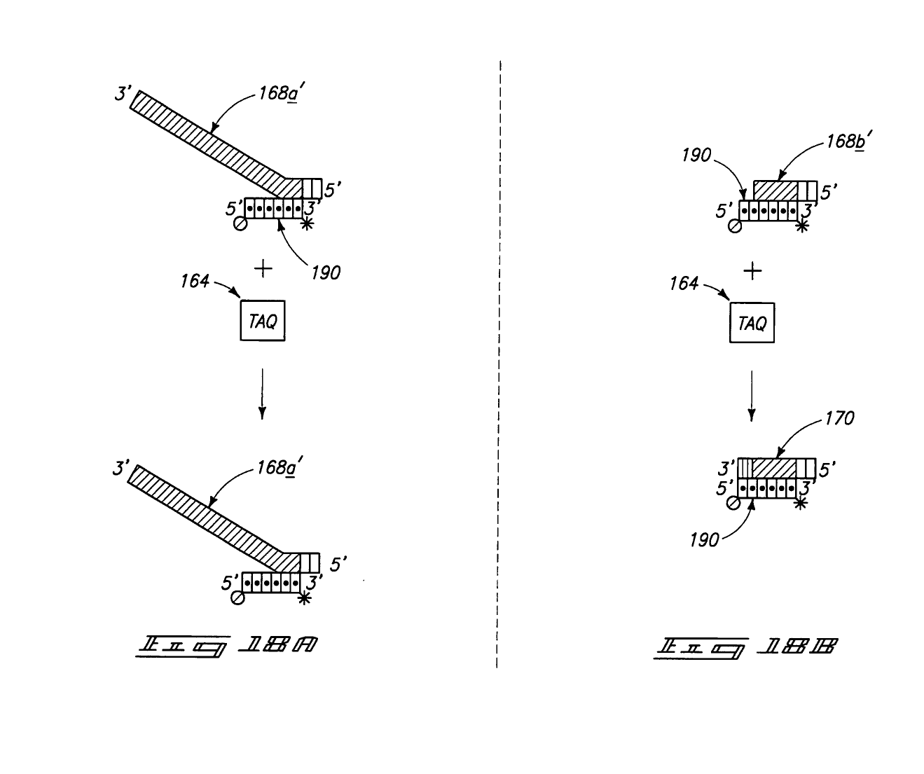

Continuing to FIG. 18, as illustrated in Panel A mismatched/non-complementary sequence between probe 190 and extension molecule 168a′ prevents hybridization along these portions of sequence and does not provide a recognition sequence or substrate function for Taq polymerase activity. Referring to Panel B, the shorter extension molecule fragment 168b′ can act as a primer for primer extension by Taq polymerase 164 resulting in a further extended molecule 170 utilizing probe 190 as a template.

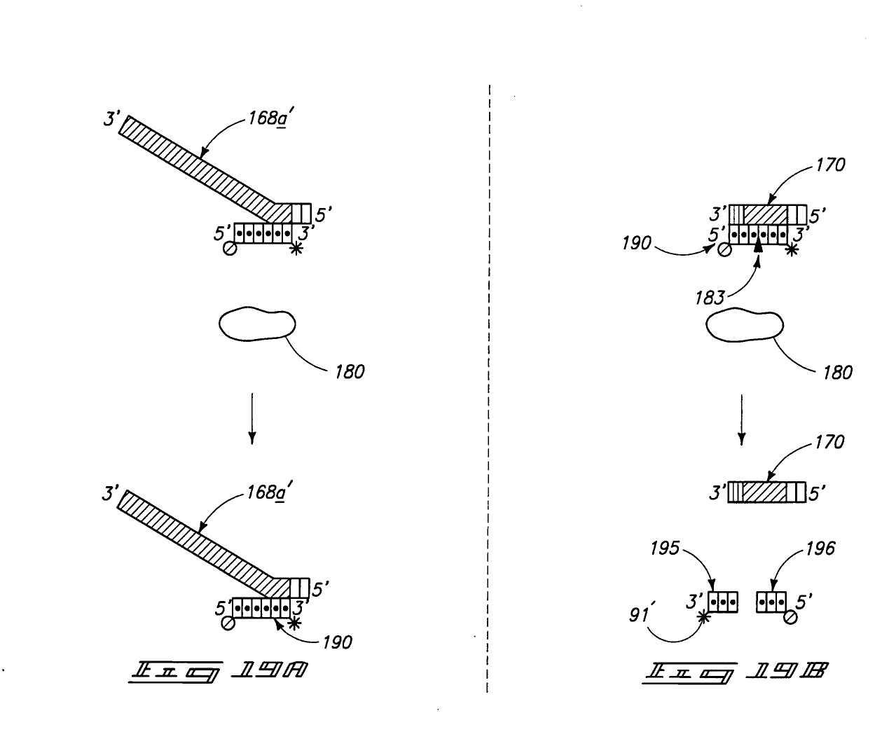

Referring to FIG. 19 Panel B, the duplex DNA molecule containing the further extended product 170 and probe 190 is specifically designed to provide a recognition site for endonuclease 180 and a target site 183 for cleavage by the endonuclease. As illustrated, cleavage of the probe by endonuclease 180 produces probe fragments 195 and 196 which are able to dissociate from extension product 170 thereby decreasing quenching to produce fluorescence 191 in the assay sample. It is to be noted that additional probe molecules can hybridize to the same extension molecule 170 to form a duplex recognition site and production of additional cleaved probe fragments to further amplify the signal.

In contrast, referring to FIG. 19 Panel A, incomplete hybridization of probe 190 with the full length extension fragment 168a′ does not form the endonuclease recognition site and probe 190 is therefore not cleaved when bound to product 168a (resulting from the intact template molecule 150).

The 2-step isothermal assay format was examined as set forth in the following examples.

Two-step amplification reactions were performed including an initial N-glycosylase reaction step conducted in PCR tubes in 5 μl volumes at 30-37° C. utilizing 2.5 pmol substrate oligonucleotide as set forth in SEQ ID NO.:12. The processing was conducted in an absence of N-glycosylase. Accordingly, a control oligonucleotide having SEQ ID NO.:13 was designed to mimic an abasic product that had undergone abiotic hydrolysis at the abasic site. Substrate oligonucleotide (SEQ ID NO.:12) and abasic mimic oligonucleotide SEQ ID NO.:13 were mixed at varying ratios to total 2.5 pmol “substrate” oligonucleotide in the assay mixture.

After performing the initial reactions under N-glycosylase activity conditions, reagents for signal amplification are added (50 μl) to the same tube in which the N-glycosylase reaction process was conducted. The amplification reagent mixture contains, per 1000 μl: 805 μl nuclease-free water; 100 μl 10× NEB buffer 2; 20 μl dNTP mix (as described above); 20 μl Taq polymerase (5 unites/μl); 10 μl Nb.BbvC I (10 units/μl); 20 μl primer (100 pmol/μl); and 25 μl probe (200 pmol/μl). The primer utilized contained sequence as set forth in SEQ ID NO.:14. The probe utilized contained a 5′ fluorescein label and a 3′ BHQ1 quencher label with an oligonucleotide sequence as set forth in SEQ ID NO.:15. The second stage of the two-level (signal amplification) assay was conducted at 50° C. for 10 minutes followed by 94° C. for 2 minutes to stop the endonuclease reactions. Images are obtained utilizing a UV light box.

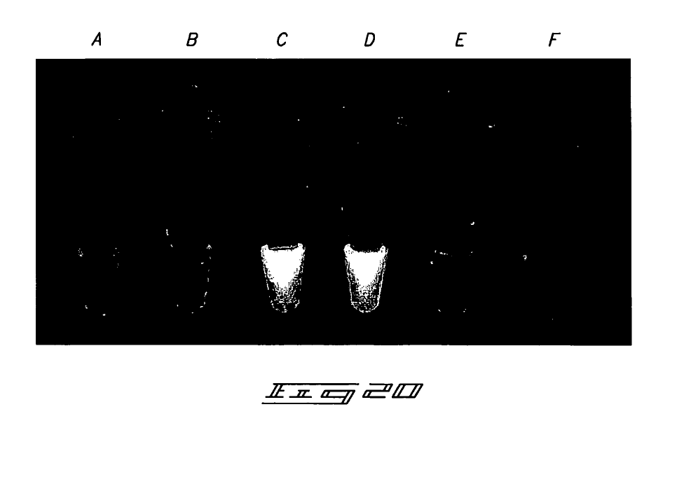

Referring to FIG. 20, control reactions using products mimics were performed with respect to the 2-step isothermal assay. In samples A and B a synthetic oligonucleotide was utilized having sequence as set forth in SEQ ID NO.:5. Such sequence mimics the elongation product produced from an intact substrate template after cleaving of the primer portion (corresponding to fragment 168a′ in FIG. 16 Panel A). Control samples 3 and 4 were performed utilizing a synthetic oligonucleotide product mimic having sequence as set forth in SEQ ID NO.:6. Such oligonucleotide mimics the short extension product after primer cleavage (corresponding to molecule 168b′ illustrated in FIG. 16 Panel B). The observable fluorescence in such samples indicates ability of such oligonucleotide to hybridize to the probe sequence to form an intact recognition site allowing cleavage of probe and increased fluorescence. Samples E and F were performed utilizing the probe in an absence of complementary oligonucleotide.

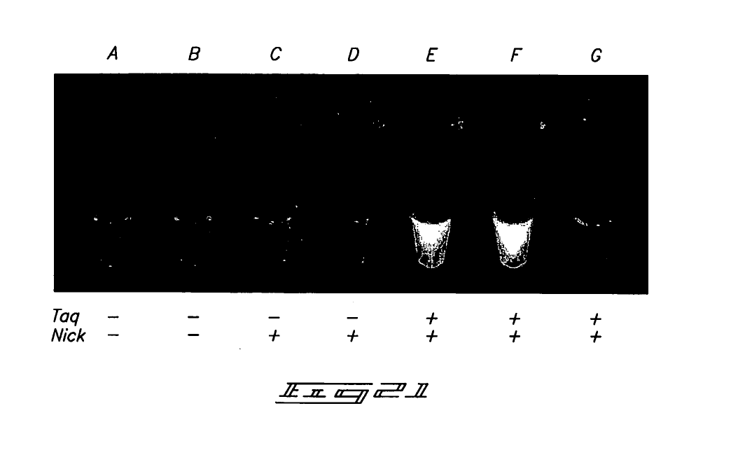

Referring to FIG. 21, such presents the results of control reactions performed utilizing a product mimic oligonucleotide having the sequence set forth in SEQ ID NO.: 7. Such sequence mimics the short extension product after cleaving primer portion 162 (corresponding to fragment 168b′ illustrated in FIG. 17B). Each of samples A-F were performed utilizing 2.5 pmol of the product mimic oligonucleotide per reaction. Sample G was performed in an absence of the product mimic oligonucleotide. Samples A-D were performed in an absence of polymerase, either in an absence of nicking endonuclease (samples A and B) or the presence of nicking endonuclease (samples C and D). Samples E and F contained both Taq polymerase and nicking endonuclease. The increase in fluorescence in samples 5 and 6 indicates successful hybridization and elongation of the product mimic along with subsequent cleavage of the probe. Control sample G was performed in the presence of both Taq polymerase and the endonuclease.

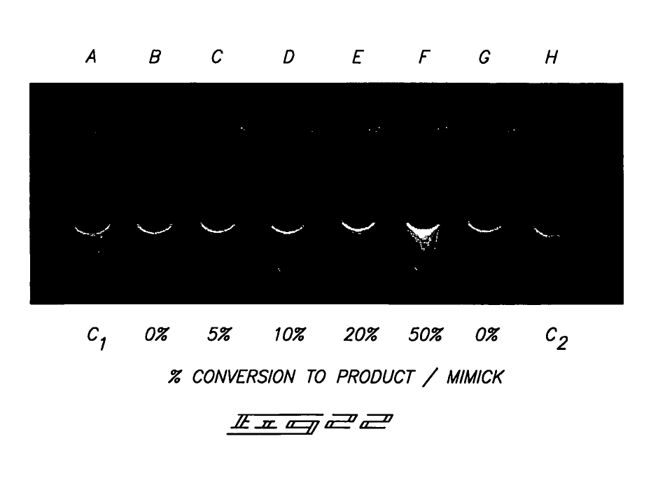

Referring to FIG. 22, such shows the results of test studies of isothermal amplification reactions using product mimics. Samples 1 and 8 are control samples containing no substrate or product. Samples B-G each contained total amount of oligonucleotides (ricin substrate polynucleotide having sequence set forth in SEQ ID NO.:12; and product mimicking oligonucleotide which mimics product hydrolyzed at the abasic site and having the sequence set forth in SEQ ID NO.:13). The percentages indicated correspond to the amount of product mimic relative to the total 2.5 pmol of ricin substrate and product mimic oligonucleotide. Primer utilized for conducting the isothermal amplification (2-step) reaction was as set forth in SEQ ID NO.:14. The probe utilized had a 5′ fluorescein label and a 3′ BHQ1 label and had an oligonucleotide sequence as set forth in SEQ ID NO.: 15. To the 2.5 pmol total oligonucleotides (volume less than 5 μl) was added 50 μl combined reagents including NEB buffer, dNTPs, Taq polymerase, Nb.BbvC I endonuclease, along with the indicated primer and probe. The reactions were then incubated for 10 minutes at 50° C. followed by a 2 minute exposure to 94° C. The samples were subsequently placed on a UV light box to obtain the image present in the figure.

The combined studies of the 2-step isothermal cascade amplification methodology set forth above indicate successful highly sensitive detection of N-glycosylase activity. In addition to being a single-tube fieldable assay, the isothermal cascade amplification implementation overcomes the need for multiple reagent additions and minimizes energy requirements due to isothermal mechanism (50° C. constant temperature). The 2-step embodiment can utilize the same enzymes as the previously described 3-level assay but is advantageously faster with similar sensitivity. The 2-step assay technique can also be utilized concurrently with antibody-based strategies for concentrating and purifying toxins from samples.

The methodology described above can be advantageously utilized for bio-defense applications. The assays are relatively simple and robust relative to conventional assay methodology. Such assays are potentially useful for all DNA glycosylases regarded as bio-threats in natural forms as well as engineered toxins.

The invention additionally encompasses N-glycosylase detection kits. In general the N-glycosylase detection kits of the invention will include a substrate polynucleotide having an N-glycosylase target sequence as described above. Such kits can further include an oligonucleotide primer for use during polymerase reactions as described above. Kits can further comprise a probe for utilization in signal amplification/detection. Typically the probe will comprise an oligonucleotide labeled with a first and second label such as described above. A polymerase and/or a DNA nicking enzyme such as those described above can be provided in the kit or can be obtained separately. The particular substrates, probes and primers can be of specific design for performing a 3-level assay or for performing a 2-step assay as described above. Where the kit is to be utilized in a remote or in-field location, the kit can preferably contain all buffer constituents for performing the respective assay.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

The invention claimed is:

1. A method of detecting the presence of a glycosylase, comprising:
providing a sample to be tested for the presence of a glycosylase;
mixing the sample with a substrate polynucleotide to form an initial mixture;
adding an oligonucleotide primer and a polymerase to the initial mixture to form an assay mixture resulting in extended primer molecules;
providing an endonuclease into the assay mixture; and
contacting the assay mixture with a probe comprising a probe oligonucleotide sequence labeled with a first label and a second label.

2. The method of claim 1 further comprising subjecting the assay mixture to thermocycling prior to providing the endonuclease.

3. The method of claim 1 wherein the endonuclease is provided into the assay mixture at the time of adding the polymerase.

4. The method of claim 1 wherein the first label comprises a fluorophore and the second label comprises a fluorescence quencher.

5. The method of claim 1 wherein the extended primer molecule is capable of forming an intramolecular hairpin loop.

6. The method of claim 1 wherein the first label is at the 5′-end of the oligonucleotide and the second label is at the 3′-end of the oligonucleotide.

7. The method of claim 1 wherein glycosylase activity produces a detectable increase in fluorescence relative to an absence of glycosylase activity.

8. The method of claim 1 wherein the substrate polynucleotide consists of a single strand DNA molecule having at least one base capable of removal by glycosylase activity to produce an abasic site, wherein primer extension activity of the polymerase is inhibited by the abasic site, and wherein inhibition of the polymerase at the abasic site results in production of an extended signal oligonucleotide sequence which contains fewer nucleotides than the substrate polynucleotide and comprises a first extension portion, a complementary portion which is complementary to the first extension portion, and a template portion that can serve as a template for further extension of the signal oligonucleotide by the polymerase to produce a second extension portion.

9. The method of claim 8 wherein extension of the signal oligonucleotide by the polymerase produces a first and a second recognition site for the endonuclease, and wherein the endonuclease cleaves within the template portion after extension to expose a single-stranded second extension portion.

10. The method of claim 9 wherein after endonuclease cleavage of the template portion, the probe hybridizes to the exposed second extension portion.

11. The method of claim 10 wherein the probe oligonucleotide comprises SEQ ID NO.: 9.

12. The method of claim 10 wherein hybridization of the probe and the second extension portion produces a third recognition site for the endonuclease.

13. The method of claim 12 wherein cleaving of the probe by the endonuclease results in a decrease in fluorescence quenching and a regenerated second extension portion.

14. The method of claim 1 wherein the endonuclease is a nicking endonuclease that cleaves only one strand of DNA on a double-stranded DNA substrate.

15. The method of claim 1 wherein the substrate polynucleotide consists of a single strand DNA molecule having at least one base capable of removal by glycosylase activity to produce an abasic site, wherein primer extension activity of the polymerase is inhibited by the abasic site, and wherein inhibition of the polymerase at the abasic site results in production of an extended signal oligonucleotide sequence which contains fewer nucleotides than the substrate polynucleotide and comprises a portion complementary to at least a portion the probe oligonucleotide.

16. The method of claim 15 wherein the probe oligonucleotide comprises SEQ ID NO.:15

17. An N-glycosylase assay method comprising:
providing a sample to be tested for N-glycosylase activity;
mixing the sample with substrate polynucleotide molecules; and
detecting the presence of abasic sites produced on the polynucleotide molecules, the detecting comprising:
producing an oligonucleotide product complementary to a portion of the substrate polynucleotide sequence ending at the abasic site;
dissociating the oligonucleotide product from the substrate polynucleotide;
extending the oligonucleotide product utilizing a polymerase;
hybridizing a probe to a portion of the oligonucleotide product; and
cleaving the probe.

18. The assay method of claim 17 wherein the probe is hybridized to a portion of the oligonucleotide product prior to the extending.

19. The assay method of claim 17 wherein the extending occurs prior to the hybridizing.

20. The assay method of claim 17 wherein the probe comprises a fluorescent label and a quenching label, and wherein the cleaving the probe produces an increase in detectible fluorescence.

21. The assay method of claim 17 wherein the probe comprises a nucleic acid oligomer having from 14 nucleotides to 40 nucleotides.

22. The assay method of claim 17, wherein the producing the oligonucleotide product comprises providing a reverse primer which is extended to produce an extended reverse primer, and wherein the extended reverse primer is cleaved by an endonuclease prior to the dissociating the oligonucleotide product from the substrate polynucleotide.

23. The assay method of claim 17, wherein the replicating comprises providing a reverse primer which is extended to produce an extended reverse primer, and wherein the extended reverse primer is cleaved by an endonuclease after the dissociating the oligonucleotide product from the substrate polynucleotide.

24. The assay method of claim 17 wherein the substrate polynucleotide molecules comprise a recognition sequence recognizable by one or more N-glycosylases.

25. The assay method of claim 17 wherein the recognition sequence is substantially specific to a particular N-glycosylase.

26. The assay method of claim 17 wherein the producing the oligonucleotide product is performed within an assay mixture, and wherein the dissociating the oligonucleotide product comprises heating the assay mixture.

27. An oligonucleotide probe comprising an oligonucleotide sequence selected from SEQ ID NO.:9 and SEQ ID NO.:15.

28. The oligonucleotide probe of claim 27 further comprising a first label proximate the 5′-end of the oligonucleotide sequence, and a second label proximate the 3′-end of the oligonucleotide sequence.

29. The oligonucleotide probe of claim 28 wherein one of the first and second labels is a fluorescent label and the other is a quencher.

30. A synthetic polynucleotide comprising the sequence set forth in SEQ ID NO.:1.

31. The synthetic polynucleotide of claim 30 wherein the nucleotides at positions 6 and 15 are complementary relative to one another, wherein the nucleotides at positions 7 and 14 are complementary relative to one another, and wherein the nucleotides at positions 8 and 13 are complementary relative to one another.

32. A composition of matter comprising a template oligonucleotide comprising SEQ ID NO.:1 and a transcription primer comprising SEQ ID.NO.:2.

33. The composition of matter of claim 32 wherein the nucleotide at position 6 of SEQ ID NO.:2 is mismatched with respect to the nucleotide at position number 32 of SEQ ID NO.:1.

34. The composition of matter of claim 32 wherein nucleotides 20-31 of SEQ ID NO.:1 are complementary to nucleotides 18-7 of SEQ ID NO.:2.

35. An N-glycosylase detection kit comprising:
a substrate polynucleotide having an N-glycosylase target sequence;
a DNA endonuclease; and
a probe comprising a fluorescent label at a first end of a probe oligonucleotide and a quencher moiety at a second end of the oligonucleotide.

36. The kit of claim 35 wherein the first end is the 5′-end of the probe oligonucleotide.

37. The kit of claim 35 further comprising a polymerase.

38. The kit of claim 35 wherein the probe oligonucleotide comprises a sequence selected from SEQ ID NO.:9 and SEQ ID NO.:15.

39. The kit of claim 35 wherein the substrate polynucleotide comprises a sequence selected from SEQ ID NOs.:1, 4, and 12.

40. The kit of claim 35 wherein the endonuclease is a site specific and strand specific endonuclease having a recognition sequence on double-stranded DNA.

41. The kit of claim 35 further comprising a reverse primer comprising a sequence complementary to a portion of the substrate polynucleotide sequence.

42. The kit of claim 41 wherein the reverse primer sequence comprises a nicking site for the endonuclease, wherein the endonuclease has a duplex DNA recognition sequence, and wherein the duplex DNA recognition sequence is present in a hybridized complex of an extended version of the reverse primer and the substrate polynucleotide.


SEQUENCE LISTING (17 sequences)

SEQ ID NO: 1 - DNA, 32 bp, Artificial
  synthetic oligonucleotide
tttttsssgagasssbbbbbbbbbbbbbbbbn

SEQ ID NO: 2 - DNA, 18 bp, Artificial
  synthetic oligonucleotide
nnnnnnvvvvvvvvvvvv

SEQ ID NO: 3 - DNA, 32 bp, Artificial
  synthetic sequence
ttttgccgngaggctccggtcggttgtttggn

SEQ ID NO: 4 - DNA, 32 bp, Artificial
  synthetic
ttttgccgagaggctccggtcggttgtttggn

SEQ ID NO: 5 - DNA, 20 bp, Artificial
  synthetic
tgagggcaagcctctcggcc

SEQ ID NO: 6 - DNA, 23 bp, Artificial
  synthetic
tgagggcaagcctcagcaagaag

SEQ ID NO: 7 - DNA, 14 bp, Artificial
  synthetic
tgagggcaagcctc

SEQ ID NO: 8 - DNA, 38 bp, Artificial
  synthetic oligonucleotide
cttcttgctgaggtgctgaggctccaaacaaccgaccg

SEQ ID NO: 9 - DNA, 16 bp, Artificial
  synthetic oligonucleotide
cttcttgctgaggtgc

SEQ ID NO: 10 - DNA, 32 bp, Artificial; identical to SEQ ID NO: 3
  synthetic oligonucleotide with abasic site

SEQ ID NO: 11 - DNA, 23 bp, Artificial
  synthetic oligonucleotide
gaggctccggtcggttgtttggn

SEQ ID NO: 12 - DNA, 38 bp, Artificial
  synthetic
ggccgagaggcttgccctcagctctttctgtctttctt

SEQ ID NO: 13 - DNA, 32 bp, Artificial
  synthetic oligonucleotide
gaggcttgccctcagctctttctgtctttctt

SEQ ID NO: 14 - DNA, 20 bp, Artificial
  synthetic oligonucleotide
aagaaagacagaaagagctg

SEQ ID NO: 15 - DNA, 22 bp, Artificial
  synthetic oligonucleotide
cttcttgctgaggcttgccctc

SEQ ID NO: 16 - DNA, 38 bp, Artificial
  synthetic oligonucleotide
tgaggctccaaacggagcctcagcacctcagcaagaag

SEQ ID NO: 17 - DNA, 46 bp, artificial
  synthetic oligonucleotide
tgaggctccaaacaaccgaccggagcctcagcacctcagcaagaag